(19) United States
(12) Patent Application Publication
(10) Pub. No.: US 2022/0401143 A1
(43) Pub. Date: Dec. 22, 2022

(54) ELECTROSURGICAL SYSTEMS AND METHODS

(71) Applicant: Covidien LP, Mansfield, MA (US)

(72) Inventor: Grant T. Sims, Boulder, CO (US)

(21) Appl. No.: 17/702,629

(22) Filed: Mar. 23, 2022

Related U.S. Application Data
(60) Provisional application No. 63/211,257, filed on Jun. 16, 2021.

(51) Int. Cl. A61B 18/14; G06N 20/00; A61B 34/30

(52) U.S. Cl. CPC A61B 18/1445; G06N 20/00; A61B 34/30; A61B 2018/0063

(57) ABSTRACT

A method of sealing tissue includes attempting to grasp tissue between first and second jaw members of an end effector assembly of an electrosurgical instrument, attempting to conduct electrosurgical energy between the first and second jaw members, and determining, based on impedance feedback from the electrosurgical energy, whether an error exists. In a case where no error is detected, the method includes implementing a tissue treating algorithm to treat tissue grasped between the first and second jaw members. The tissue treating algorithm includes conducting electrosurgical energy between the first and second jaw members and through tissue grasped therebetween. In a case where an error is detected, the method includes determining, based on additional feedback data, a cause of the error, and outputting an alarm indicating the error and the cause of the error.

### CROSS-REFERENCE TO RELATED APPLICATIONS

This application claims the benefit of, and priority to, U.S. Provisional Patent Application No. 63/211,257, filed on Jun. 16, 2021, the entire contents of which are hereby incorporated herein by reference.

### FIELD

The present disclosure relates to electrosurgery and, more particularly, to electrosurgical systems and methods for detecting errors and causes thereof to facilitate safe and effective tissue sealing.

### BACKGROUND

In bipolar electrosurgery, electrical current is conducted through tissue positioned between electrodes of different polarity to heat and thereby treat the tissue. Bipolar electrosurgery often involves the use of an electrosurgical forceps, a pliers-like instrument that relies on mechanical action between its jaws to grasp, clamp, and constrict tissue. Electrosurgical forceps, more specifically, utilize mechanical clamping action and electrical energy to treat, e.g., cauterize, coagulate, and/or seal, clamped tissue.

Whereas cauterization involves the use of heat to destroy tissue and coagulation is a process of desiccating tissue such that the tissue cells are ruptured and dried, tissue sealing is a process of liquefying the collagen, elastin, and ground substances in the tissue so that they reform into a fused mass with significantly reduced demarcation between opposing tissue structures. In order to create an effective tissue seal, two predominant mechanical parameters must be accurately controlled: the pressure applied to the tissue and the gap distance between the electrodes. In addition, electrosurgical energy must be applied to the tissue under controlled conditions, e.g., controlling the intensity, frequency, and duration of electrosurgical energy application to tissue, to ensure creation of an effective tissue seal.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is a method of sealing tissue including attempting to grasp tissue between first and second jaw members of an end effector assembly of an electrosurgical instrument, attempting to conduct electrosurgical energy between the first and second jaw members, and determining, based on impedance feedback from the electrosurgical energy, whether an error exists. In a case where no error is detected, the method further includes implementing a tissue treating algorithm to treat tissue grasped between the first and second jaw members. The tissue treating algorithm includes conducting electrosurgical energy between the first and second jaw members and through tissue grasped therebetween. In a case where an error is detected, the method further includes determining, based on additional feedback data, a cause of the error, and outputting an alarm indicating the error and the cause of the error.

In an aspect of the present disclosure, the additional feedback data includes electrical feedback data from the electrosurgical energy.

In another aspect of the present disclosure, the additional feedback data includes sensor data obtained from a sensor. In such aspects, the sensor may be configured to one of: sense a physical property of tissue, sense a physical property of the end effector assembly, visualize tissue, or visualize the end effector assembly.

In still another aspect of the present disclosure, in a case where the error is detected, the method further includes determining, based on the cause of the error, whether the error is recoverable.

In yet another aspect of the present disclosure, in a case where the error is determined to be recoverable, the method further includes enabling electrosurgical energy to be subsequently conducted between the first and second jaw members. In a case where the error is determined not to be recoverable, the method further includes inhibiting the supply of electrosurgical energy to the first and second jaw members.

In still yet another aspect of the present disclosure, the method further includes determining the cause of the error by selecting the cause of the error from a debug list based upon the additional feedback data. The selecting may be performed, in aspects, by a machine learning program. In aspects, the debug list includes: a tissue grasping error, a field error, and an instrument error. Additional or alternative items on the debug list, a customized debug list, an updatable debug list, etc. are also contemplated. Depending upon the error, the supply of energy may be stopped or may be allowed to continue as-is or only after modification of energy-delivery, the instrument, etc. (automatically or by the user).

In another aspect of the present disclosure, the attempting to conduct the electrosurgical energy between the first and second jaw members includes attempting to conduct an interrogation signal between the first and second jaw members. The interrogation signal may include at least one pulse and/or may be a constant-power signal.

An electrosurgical system provided in accordance with the present disclosure includes an end effector assembly including first and second jaw members at least one of which is movable relative to the other from a spaced-apart position to an approximated position for grasping tissue therebetween. The system further includes an electrosurgical generator configured to supply electrosurgical energy to the first and second jaw members for conduction therebetween and through grasped tissue to treat the grasped tissue. The electrosurgical generator includes a processor and memory storing instructions that, when executed by the processor, cause the processor to determine, based on impedance feedback from the electrosurgical energy, whether an error exists. In a case where no error is detected, the processor is caused to implement a tissue treating algorithm to treat tissue grasped between the first and second jaw members, e.g., conducting electrosurgical energy between the first and second jaw members and through tissue grasped therebetween. In a case where an error is detected, the processor is caused to determine, based on additional feedback data, a cause of the error, and output an alarm indicating the error and the cause of the error.

In an aspect of the present disclosure, the end effector assembly is disposed at a distal end of a handheld surgical instrument. Alternatively, the end effector assembly may be coupled to a robotic surgical system.

In another aspect of the present disclosure, the additional feedback data includes electrical feedback data from the electrosurgical energy. In alternative or additional aspects, the additional feedback data includes sensor data obtained from a sensor associated with the end effector assembly.

In still another aspect of the present disclosure, in a case where the error is detected, the processor is further caused to determine, based on the cause of the error, whether the error is recoverable. In a case where the error is determined to be recoverable, the processor is further caused to enable electrosurgical energy to be subsequently supplied from the electrosurgical generator to the end effector assembly. In a case where the error is determined not to be recoverable, the processor is further caused to inhibit the supply of electrosurgical energy from the electrosurgical generator to the end effector assembly. In aspects, it is additionally or alternatively determined whether the error is a user (operation) error, an instrument error, a generator error, combinations thereof, etc.

In yet another aspect of the present disclosure, the electrosurgical generator is configured to supply electrosurgical energy to the first and second jaw members by way of an interrogation signal to enable the processor to determine whether an error exists.

In still yet another aspect of the present disclosure, the interrogation signal is at least one of: a pulse signal or a constant-power signal.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an electrosurgical system in accordance with the present disclosure including an electrosurgical forceps and an electrosurgical generator;

FIGS. 2A and 2B are enlarged, perspective views of a distal end portion of the forceps of FIG. 1 with an end effector assembly thereof disposed in spaced-apart and approximated positions, respectively;

FIG. 3 is a schematic illustration of a robotic surgical system configured for use in accordance with the present disclosure;

FIG. 4 is a block diagram of the generator of FIG. 1;

FIG. 5 is a longitudinal, cross-sectional view of another end effector assembly configured for use with the forceps of FIG. 1 or the system of FIG. 3;

FIG. 6 is a transverse, cross-sectional view of the end effector assembly of FIG. 5 with a sensor(s) separate from the end effector assembly;

FIG. 7 is a block diagram of a generator configured for use with the end effector assemblies of FIGS. 5 and 6;

FIG. 8 is a logic diagram of a machine learning algorithm in accordance with the present disclosure; and

FIG. 9 is a flow diagram of a method provided in accordance with the present disclosure.

### DETAILED DESCRIPTION

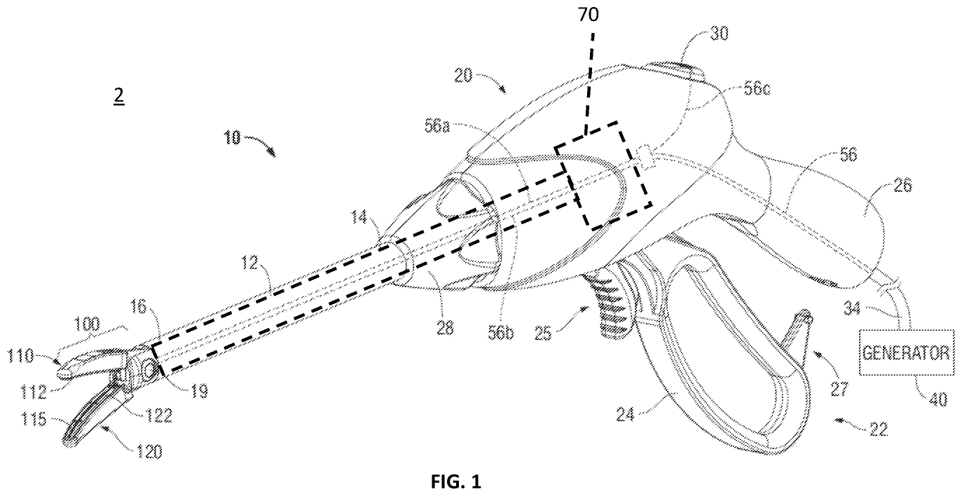

Referring to FIG. 1, an electrosurgical system in accordance with the present disclosure is shown generally identified by reference numeral 2. Electrosurgical system 2 includes an electrosurgical forceps 10 and an electrosurgical generator 40. Electrosurgical forceps 10 is shown and described herein as a shaft-based, manual device. However, any other suitable electrosurgical forceps, whether shaft-based, hemostat-style, manual, partly powered, fully powered, robotic, etc. may be utilized in accordance with the present disclosure. Obviously, different connections and considerations apply to each particular type of instrument; however, the aspects and features of the present disclosure with respect to sealing tissue remain generally consistent with respect to any suitable instrument.

Continuing with reference to FIG. 1, forceps 10 includes a shaft 12, a housing 20, a handle assembly 22, a trigger assembly 25, a rotating assembly 28, and an end effector assembly 100. Shaft 12 has a distal end portion 16 configured to mechanically engage end effector assembly 100 and a proximal end portion 14 that mechanically engages housing 20. A cable 34 couples forceps 10 to electrosurgical generator 40 for transmitting energy and control signals between generator 40 and forceps 10. Cable 34 houses a plurality of wires 56 that are internally divided within handle assembly 22 and/or in shaft 12 into wires 56a-56c, which electrically interconnect end effector assembly 100, activation switch 30, and/or generator 40 with one another. Alternatively, forceps 10 may be configured as a cordless device including power and generator functionality supported on or within housing 20.

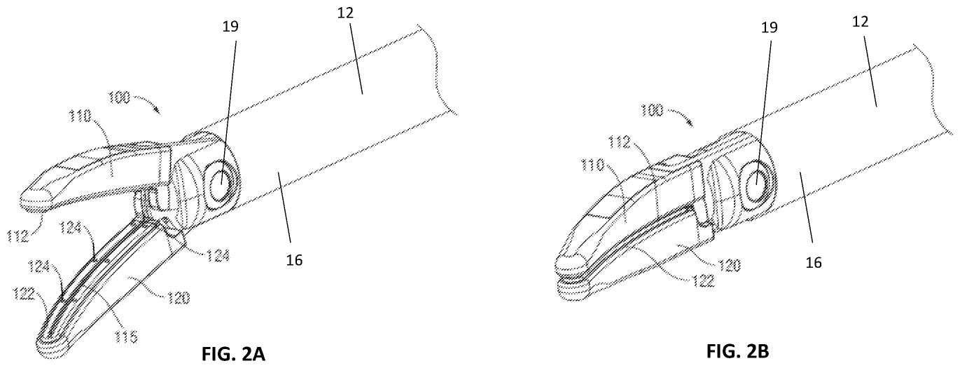

Handle assembly 22 includes a movable handle 24 and a fixed handle 26. Fixed handle 26 is integrally associated with housing 20 and movable handle 24 is movable relative to fixed handle 26. Movable handle 24 is ultimately connected to a drive assembly 70 that, together, mechanically cooperate to impart movement of one or both of jaw members 110, 120 of end effector assembly 100 relative to the other between a spaced-apart position and an approximated position to grasp tissue therebetween. As shown in FIG. 1, movable handle 24 is initially spaced-apart from fixed handle 26 and, correspondingly, jaw members 110, 120 are disposed in the spaced-apart position (see also FIG. 2A). Movable handle 24 is movable from this initial position to one or more compressed positions corresponding to one or more approximated positions of jaw members 110, 120 (see FIG. 2B).

Drive assembly 70 may be configured to regulate the clamping force applied to tissue grasped between surfaces 112, 122 of jaw members 110, 120, respectively. More specifically, handle assembly 22 and/or latching assembly 27, in conjunction with drive assembly 70, may be configured such that jaw members 110, 120 impart a specific clamping force or clamping force within a specific clamping force range to tissue grasped between surfaces 112, 122 of jaw members 110, 120, respectively. This may be achieved manually, e.g., via moving movable handle 24 from the initial position to a specific compressed position (or positions), e.g., a fully compressed position; via mechanical latching, e.g., wherein a latch assembly 27 is configured to latch movable handle 24 in a specific position (or positions); via a powered actuator with feedback-based control, e.g., via driving or reversing a motor-controlled actuator to a specific position (or positions); and/or via any other suitable mechanism. Drive assembly 70, in any of the configurations detailed above or any other suitable configuration, may include one or more passive regulating components, e.g., springs, resilient features, etc., and/or active regulating components, e.g., motor(s), manual drives, etc.

Suitable mechanisms for use as or in conjunction with drive assembly 70 for clamping force control include those described in U.S. Pat. Nos. 5,776,130; 7,766,910; 7,771,426; and 8,226,650; and/or U.S. Patent Application Pub. Nos. 2009/0292283; 2012/0172873; and 2012/0184988, the entire contents of all of which are hereby incorporated by reference herein. Other suitable mechanisms for applying a specific clamping force or clamping force within a specific clamping force range to tissue grasped between jaw members 110, 120 may also be provided. With tissue grasped between jaw members 110, 120 under the specific clamping force or clamping force within a specific clamping force range, energy may be supplied to either or both tissue contacting surfaces 112, 122 of jaw members 110, 120, respectively, to seal tissue, e.g., via activation of activation switch 30.

The jaw clamping force, measured at a midpoint along the lengths of jaw members 110, 120, may be in a range of (or the jaw force range may be) from about 7.0 lbf to about 11.0 lbf; in other aspects from about 8.0 lbf to about 10.0 lbf; and, in still other aspects, from about 8.5 lbf to about 9.5 lbf.

Latching assembly 27 may be provided for selectively locking movable handle 24 relative to fixed handle 26 at various positions between the initial position and the compressed position(s) to correspondingly lock jaw members 110, 120 at various different positions during pivoting, e.g., the one or more approximated positions. Rotating assembly 28 is rotatable in either direction to similarly rotate shaft 12 and end effector assembly 100 relative to housing 20.

Referring also to FIGS. 2A and 2B, end effector assembly 100 is shown attached at the distal end portion 16 of shaft 12 and includes opposing jaw members 110 and 120. Each jaw member 110, 120 includes an electrically conductive tissue contacting surface 112, 122, respectively, that cooperate to grasp tissue therebetween, e.g., in the one or more approximated positions of jaw members 110, 120, and to facilitate sealing the grasped tissue via conducting the energy from generator 40 therebetween. More specifically, tissue contacting surfaces 112, 122 are electrically coupled to generator 40, e.g., via wires 56a, 56b, and are configured to be energized to different potentials to enable the conduction of Radio Frequency (RF) electrosurgical energy provided by generator 40 between tissue contacting surfaces 112, 122 and through tissue grasped therebetween to seal tissue. Tissue contacting surfaces 112, 122 may be defined by electrically conductive plates secured to jaw members 110, 120, may be defined by surfaces of jaw members 110, 120 themselves, may be formed via the deposition of material onto jaw members 110, 120, or may be defined and/or formed in any other suitable manner.

Either or both jaw members 110, 120 may further include one or more stop members 124 (FIG. 2A) disposed on or otherwise associated with either or both tissue-contacting surface 112, 122 to maintain a minimum gap distance between tissue contacting surfaces 112, 122 when jaw members 110, 120 are disposed in a fully approximated position, thus inhibiting electrical shorting. Stop members 124 may be insulative, partly insulative, and/or electrically isolated from either or both tissue contacting surfaces 112, 122. In aspects, in the approximated position of jaw members 110, 120, it is desirable to maintain a gap distance within a suitable gap distance range to ensure consistent and effective tissue sealing. The gap distance may be controlled by stop members 124, movable handle 24, latching assembly 27, and/or drive assembly 70, and, in aspects, may be from about 0.001 inches to about 0.010 inches; in other aspects from about 0.001 inches to about 0.008 inches; and, in still other aspects form about 0.001 inches to about 0.006 inches. Other suitable gap distance ranges are also contemplated. The gap distance may be determined as the maximum gap distance between the tissue contacting surfaces 112, 122 at any point therealong.

An activation switch 30 is disposed on housing 20 and is coupled between or otherwise to generator 40 and/or tissue-contacting surfaces 112, 122 via wire 56c. Activation switch 30 is selectively activatable to initiate the supply of energy from generator 40 to tissue contacting surfaces 112, 122 of jaw members 110, 120, respectively, of end effector assembly 100. More specifically, depression of activation switch 30 is recognized, e.g., as a resistance drop, by generator 40 to signal to generator 40 to initiate tissue sealing, e.g., to supply energy to jaw members 110, 120.

End effector assembly 100 is designed as a bilateral assembly, e.g., wherein both jaw member 110 and jaw member 120 are movable about a pivot 19 relative to one another and to shaft 12. However, end effector assembly 100 may alternatively be configured as a unilateral assembly, e.g., wherein one of the jaw members 110, 120 is fixed relative to shaft 12 and the other jaw member 110, 120 is movable about pivot 19 relative to shaft 12 and the fixed jaw member.

In some configurations, a knife assembly (not shown) is disposed within shaft 12 and a knife channel 115 is defined within one or both jaw members 110, 120 to permit reciprocation of a knife blade (not shown) therethrough, e.g., via actuation of trigger assembly 25, to mechanically cut tissue grasped between jaw members 110, 120. In aspects, the knife blade is energizable to enable dynamic energy-based tissue cutting. Alternatively, end effector assembly 100 may include a static energy-based tissue cutter (not shown), e.g., disposed one or within one of the jaw members 110, 120. The energy-based cutter, whether static or dynamic, may be configured to supply any suitable energy, e.g., RF, microwave, infrared, light, ultrasonic, thermal, etc., to tissue for energy-based tissue cutting. Energy activation for tissue cutting may be initiated via trigger assembly 25, automatically after tissue sealing, via a different (or further) activation of switch 30, via a separate actuation button, via a foot switch (not shown), or in any other suitable manner.

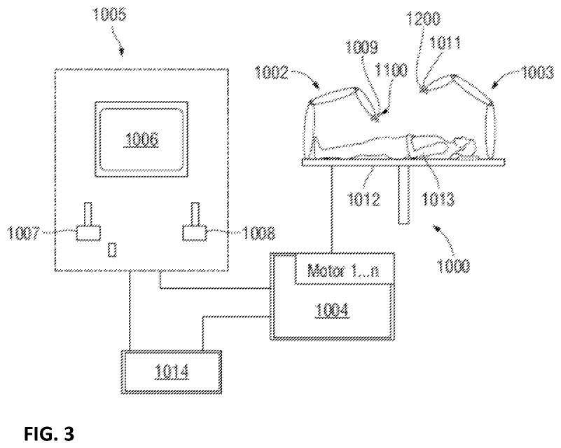

Turning to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in an operating mode. Robotic surgical instrument 1000 may be configured for use on a patient 1013 lying on a patient table 1012 to be treated in a minimally invasive manner. Robotic surgical instrument 1000 may further include or be capable of accessing a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100, for example, may be similar to and include any of the features of end effector assembly 100 (FIGS. 1-2B) and, together with robot arm 1002, functions similarly as detailed above with respect to forceps 10 except in a robotically-actuated and controlled manner. Other suitable end effector assemblies for coupling to attaching device 1009 are also contemplated. End effector assembly 1200 may be any end effector assembly, e.g., a surgical camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

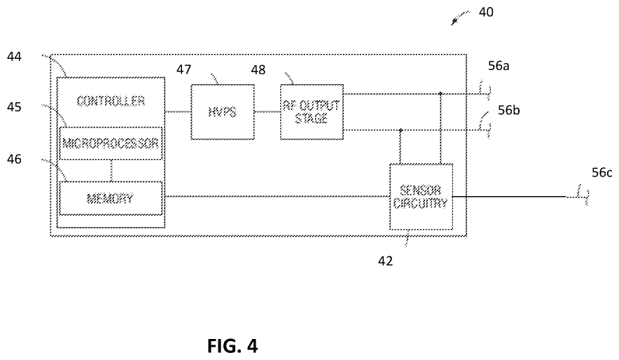

With reference to FIG. 4, generator 40 may be configured for use with forceps 10 (FIG. 1), robotic surgical system 1000 (FIG. 3), and/or any other suitable surgical instrument or system. Generator 40 includes sensor circuitry 42, a controller 44, a high voltage DC power supply (“HVPS”) 47 and an RF output stage 48. HVPS 47 provides high voltage DC power to RF output stage 48 which converts the high voltage DC power into RF energy for delivery to the end effector assembly, e.g., tissue-contacting surfaces 112, 122 of jaw members 110, 120, respectively, of end effector assembly 100 (FIGS. 1-2B). In particular, RF output stage 48 generates sinusoidal waveforms of high frequency RF energy. RF output stage 48 is configured to generate a plurality of waveforms having various duty cycles, peak voltages, crest factors, and other parameters, depending on a particular mode of operation.

Controller 44 includes a microprocessor 45 (or other suitable processor such as, for example, a digital signal processor, an ASIC, a graphics processing unit (GPU), a field-programmable gate array (FPGA), or a central processing unit (CPU)) operably connected to a memory 46 which may be volatile type memory (e.g., RAM) and/or non-volatile type memory (e.g., flash media, disk media, etc.). Microprocessor 45 is operably connected to HVPS 47 and/or RF output stage 48 allowing microprocessor 45 to control the output of generator 40, e.g., in accordance with feedback received from sensor circuitry 42. Sensor circuitry 42 is operably coupled to wires 56a, 56b, which supply energy to/from tissue-contacting surfaces 112, 122 (FIGS. 1-2B). From wires 56a, 56b and, more specifically, the signals transmitted therealong, sensor circuitry 42 may determine one or more parameters, e.g., tissue impedance, current, voltage, and/or power, etc. Sensor circuitry 42 provides feedback, e.g., based on the sensed parameter(s), to controller 44 which, in turn, selects an energy-delivery algorithm, modifies an energy-delivery algorithm, and/or adjusts energy-delivery parameters based thereon. Sensor circuitry 42 or controller 44 may also monitor wire 56c to determine activation (and/or deactivation) of switch 30 (FIG. 1) to, in response thereto, initiate (or terminate) the supply of energy based thereon.

In aspects, memory 46 can be separate from controller 44 and communicate with microprocessor 45 through communication buses of a circuit board and/or through communication cables such as serial ATA cables or other types of cables. Regardless of the location(s) of memory 46 and/or microprocessor 45, memory 46 includes computer-readable instructions that are executable by microprocessor 520 to operate controller 44, e.g., for executing various algorithms such as, for example, fixed algorithms, machine learning algorithms, etc. Controller 44 may further include a network interface (not shown) to communicate with other computers or a server. In aspects, a storage device (not shown) of controller 44 or separate therefrom may be used for storing data.

Although illustrated as part of generator 40, it is also contemplated that controller 44 be remote from generator 40, e.g., on a remote server, and accessible by generator 40 via a wired or wireless connection. In configurations where controller 44 is remote, it is contemplated that controller 44 may be accessible by and connected to multiple generators 40.

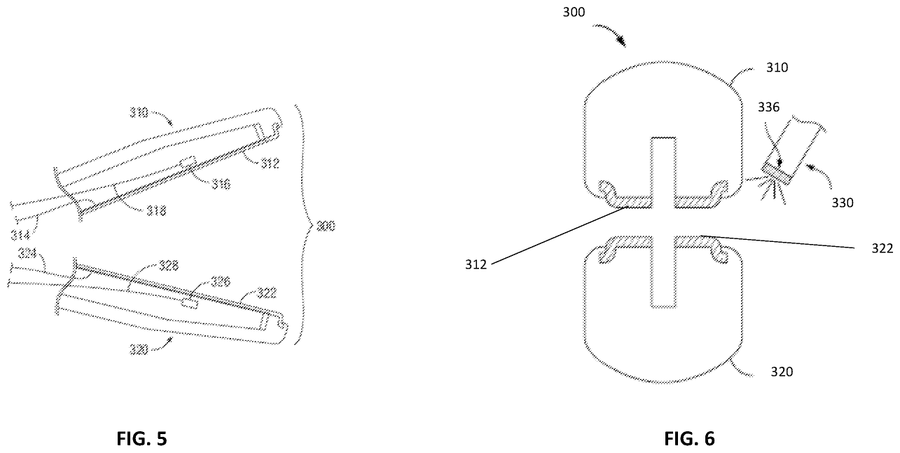

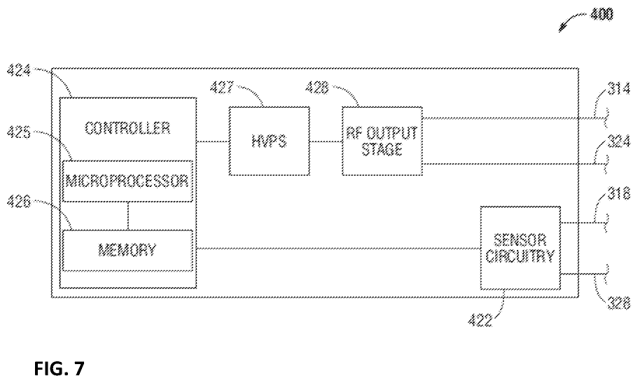

FIG. 5 illustrates another end effector assembly provided in accordance with the present disclosure, shown generally identified by reference numeral 300. End effector assembly 300 is similar to end effector assembly 100 (FIGS. 1-2B) and may include any of the features thereof. End effector assembly 300 may be utilized with forceps 10 (FIG. 1), robotic surgical system 1000 (FIG. 3), or any other suitable surgical instrument or system. End effector assembly 300 includes first and second jaw members 310, 320 each including a respective electrically conductive tissue contacting surface 312, 322. Either or both of jaw members 310, 320 is movable relative to the other between a spaced-apart position and one or more approximated positions to grasp tissue between tissue contacting surfaces 312, 322. Wires 314, 324 electrically couple tissue contacting surfaces 312, 322 to a source of energy, e.g., generator 400 (FIG. 7), for conducting RF energy between tissue contacting surfaces 312, 322 and through tissue grasped therebetween to seal tissue.

Either or both jaw members 310, 320 of end effector assembly 300 further includes a sensor 316, 326 positioned thereon or therein. Sensors 316, 326 may be any suitable sensors configured to sense one or more parameters, may sense similar or different parameters, and/or may operate independently or collectively. Sensors 316, 326 may be configured as, for example: electrical sensors, e.g., configured to sense impedance, current, power, voltage, etc.; optical sensors, e.g., configured to sense one or more optical properties of end effector assembly 300, tissue, and/or the surrounding environment such as, for example, color, clarity, transparency, etc.; distance (linear or angular) sensors configured to determine a linear distance or angle between jaw members 310, 320 and/or other components; proximity sensors, e.g., hall effect sensors, configured to determine a distance between jaw members 310, 320 and/or other components to, for example, enable determination of physical tissue properties (diameter, mass, density, compressibility, etc.); particle sensors, e.g., an ionization detector or a photoelectric detector, configured to detect smoke and/or other particles; electronic nose sensors configured to electronically sense one or more smell-based properties of tissue and/or the surrounding environment; chemical sensors, e.g., a molecular sensor, gas chromatograph, etc., configured to sense one or more chemical properties of tissue and/or the surrounding environment; moisture sensors; pressure/force sensors; density sensors; temperature and/or thermal sensors; ultrasonic sensors; audio sensors; etc. Further, combinations of sensors, e.g., two or more of the above-noted or other suitable sensors, may be utilized.

Alternatively, as shown in FIG. 6, one or more sensors 336, configured similar to any of the sensors noted above or any other suitable sensor, may be disposed on a device 330 separate from end effector assembly 300 and configured to sense one or more properties of end effector assembly 300, of tissue in contact therewith, e.g., grasped thereby, and/or of the surrounding environment. In aspects, sensor 336 is a visible image surgical camera configured to sense the position and/or angle between jaw members 310, 320. The sensor 336 may alternatively be configured as an infrared camera, thermal camera, or other suitable camera or sensor.

Referring to FIGS. 5-7, another generator 400 provided in accordance with the present disclosure is similar to generator 40 (FIG. 4) and may include any of the features thereof. Generator 400 is configured for use with end effector assembly 300 and, similarly as with generator 40 (FIG. 4), includes sensor circuitry 422, a controller 424 (including a microprocessor 425 and memory 426), an HVPS 427 and an RF output stage 428. Generator 400 is configured to supply energy to end effector assembly 300 via wires 314, 324. Sensor circuitry 422 is operably coupled to wires 318, 328 (and/or wire(s) or other wired or wireless connections to sensor 336) and is thereby configured to receive the sensed parameters from sensors 316, 326 (and sensor 336). Sensor circuitry 422 provides feedback, e.g., based on the sensed parameter(s), to controller 424 which, in turn, selects an energy-delivery algorithm, modifies an energy-delivery algorithm, and/or adjusts energy-delivery parameters based thereon. Sensor circuitry 422 may also be operably coupled to wires 314, 324 to enable determination of one or more parameters and provide feedback based thereon, similarly as detailed above with respect to sensor circuitry 42 of generator 40 (FIG. 4).

With general reference to FIGS. 1-7, sealing tissue, e.g., blood vessels, tissue including blood vessels, other tissue structures, etc., is accomplished by controlling both mechanical and electrical factors. More specifically, with respect to the mechanical factors, achieving effective and consistent tissue seals is facilitated by controlling the gap distance between the opposing electrically-conductive surfaces grasping tissue therebetween and by controlling the clamping force applied to the grasped tissue. In some instances, the electrosurgical instrument itself (such as any of the instruments, end effector assemblies, or systems detailed hereinabove) may be capable of mechanically controlling the mechanical factors within suitable ranges. The electrosurgical instrument may control the clamping force to within an appropriate clamping force range (such as those detailed above) and to control the gap distance to within an appropriate gap distance range (such as those detailed above). In such instances, a suitable tissue sealing algorithm may be implemented for controlled delivery of energy to achieve effective and consistent tissue seals.

In other instances, controlling the mechanical factors may not be capable of being consistently and effectively accomplished solely using the mechanical features of the electrosurgical instrument. Rather, in such instances, it may be necessary to pre-treat tissue, e.g., by the generator (such as any of the generators detailed hereinabove) implementing a pre-treat algorithm, in order to shrink the tissue, make the tissue more compressible, or otherwise modify the tissue such that the electrosurgical instrument is capable of achieving a clamping force and/or gap distance within the appropriate ranges thereof. Once the pre-treatment is complete, e.g., according to a pre-treatment algorithm, a suitable tissue sealing algorithm may be implemented by the generator (such as any of the generators detailed hereinabove) for controlled delivery of energy to achieve effective and consistent tissue seals.

With respect to the electrical factors, controlling the intensity, frequency, and duration of the electrosurgical energy applied to the clamped tissue are important electrical considerations for sealing tissue. A suitable tissue sealing algorithm may thus be implemented by the generator to control these electrical factors, thus facilitating tissue sealing. Tissue sealing algorithms suitable for use in accordance with the present disclosure can be found in, for example, U.S. Pat. Nos. 8,920,421, 8,147,485, and 10,617,463, the entire contents of each of which are hereby incorporated herein by reference.

Despite the above-detailed mechanical and electrical considerations that enable sealing of tissue of various different types, sizes, conditions, etc., there are instances where tissue sealing may not be capable of being performed. In such instances, for example, the generator may detect an error during an initial interrogation phase of the tissue sealing and/or pre-treatment algorithm (or any other phase or portion thereof) and, where an error is detected, provide an alarm to alert the user that there is an issue that may inhibit effective tissue sealing, and to prevent further delivery of energy for safety reasons. The initial interrogation phase (of the tissue sealing and/or pre-treatment algorithm) may be accomplished by or include impedance sensing, e.g., via one or more interrogatory impedance sensing pulses conducted between the tissue-contacting surfaces and through tissue, a continuous interrogatory impedance sensing signal conducted between the tissue-contacting surfaces, and/or any other suitable sensing signal(s). During this interrogation phase, for example, constant power may be provided to check for a short circuit, open circuit, or anomalous electrical condition (e.g., unexpected voltage, current, power, impedance, changes thereof, other electrical parameter(s), etc.) in order to determine if tissue is properly grasped and capable of being sealed. More specifically, the impedance sensing signal(s) may be utilized to determine whether there is a detectable impedance (e.g., defined and non-zero), an impedance within a specified range, an impedance change, a substantially constant impedance (or insufficient impedance change), etc. Additionally or alternatively, electrical parameters may be monitored for absolute values or changes in voltage, current, power, etc. Tissue impedance (and/or other electrical parameters) during the interrogation phase is determined without appreciably changing the tissue during this phase.

If no short circuit, open circuit, or anomalous electrical condition is detected, the pre-treatment and/or tissue sealing algorithm may proceed, e.g., to subsequent phase(s), for pre-treating and/or sealing tissue. In aspects, in addition or as an alternative to detecting a short circuit, open circuit, or anomalous condition, energy delivery from a nominal energy profile can be detected and evaluated to determine whether there is an error and/or if correction to an energy delivery profile or setting is required. Even where an error condition is not detected, the results of the interrogation may dictate selection of a tissue sealing algorithm to be implemented, settings and/or parameters of the tissue sealing algorithm, whether or not pre-treatment is utilized, etc.

If a short circuit, open circuit, or anomalous condition is detected, an error is returned, and an alarm is triggered. Such an alarm may be referred to as a re-grasp alarm, as the user would be required to re-grasp tissue and re-activate the instrument to re-initiate tissue sealing. Where the error stems from, for example: too large of a bite of tissue grasped between the jaw members, an insufficient bite of tissue grasped between the jaw members, insufficient closure of the jaw members, etc., re-grasping tissue and re-activating the instrument to re-initiate tissue sealing thereafter may be sufficient to clear the alarm and enable the sealing of tissue.

However, not all errors can be remedied by re-grasping and re-activation. For example, where the error stems from an issue in the field (e.g., a wet or bloody field, a foreign object in contact with or otherwise interfering with the instrument and/or tissue to be sealed, etc.) or an issue with the instrument itself (e.g., electrical failure (due to insulation break down, wire dislodgement, etc.) and/or mechanical failure (damage to the end effector assembly, char build up on the tissue-contacting surfaces, etc.)), re-grasping tissue and re-activating the instrument will not remedy the error without further intervention. Thus, it would be beneficial to not only provide an alarm to indicate to the user that there is an error, but also to identify the cause(s) of the error and communicate the same to the user, e.g., via the alarm, to enable the user to take appropriate action, e.g., to clear the wet/bloody field, remove nearby objects, reevaluate the tissue to be sealed, perform a pre-treatment before tissue sealing, modify the tissue sealing algorithm or switch to a different tissue sealing algorithm, replace the instrument with a new instrument, etc. Further, it is noted that while the present disclosure is detailed with respect to determining an error during an interrogation phase and providing a suitable alarm or other output based thereon, it is understood that the present disclosure applies equally to determining an error at any other suitable phase or portion of a pre-treatment and/or tissue sealing algorithm and providing a suitable alarm or other output based thereon.

In order to determine the cause of the error, the generator may consider, in addition to the impedance data, other data such, as for example, by sensing or receiving data indicative of: electrical properties of tissue and/or the instrument (e.g., absolute values and/or changes in voltage, current, power, etc.); physical properties of tissue and/or the instrument (e.g., tissue type, tissue size (diameter), jaw angle, jaw force, instrument electrical defects, etc.); visualization of tissue, the instrument, and/or the surrounding environment (e.g., to determine tissue type, tissue size (diameter), jaw angle, jaw force, the presence or absence of other objections, blood, other fluids, etc., instrument electrical and/or mechanical defects, etc.); chemical analysis of tissue; optical analysis of tissue (for clarity, water content, etc.); combinations of the above; and/or any other suitable sensed or otherwise available data.

Jaw angle (and/or tissue diameter or mass based thereon), where utilized, may be determined, for example, as detailed in U.S. Pat. No. 8,357,158, and/or U.S. Patent Application Publication No. 2017/0215944, the entire contents of which are hereby incorporated herein by reference. Jaw pressure/force (and corresponding properties of tissue based thereon), where utilized, may be determined, for example, as detailed in U.S. Pat. No. 10,695,123, the entire contents of which are hereby incorporated herein by reference.

Any or all of the above sensed data may be utilized together with impedance and/or other electrical data to determine a reason for the detected error. More specifically, referring to FIGS. 4 and 7, the controller 44, 424 is configured to receive the sensor feedback from sensor circuitry 42, 422 and/or other feedback data to determine the reason for the detected error. This determination may be made, for example, by the use of one or more algorithms, set points, look-up tables, machine learning programs, etc. of which the stored or training data is obtained experimentally, via mathematical simulation, utilizing machine learning, using theoretical formulae, combinations thereof, etc. More specifically, whether provided as stored or training data, a debug list providing potential error causes (and, in aspects, sensed parameters associated threrewith), may be utilized to facilitate correlating the impedance and/or sensed data with a cause of the returned error from the debug list. The debug list may include, for example: tissue grasping/use errors (generally or specifically: too much tissue grasped, improper tissue grasping, jaw members not sufficiently closed, minimal or no tissue grasped, etc.); field errors (generally or specifically: too much blood/fluid in the field, mechanical interference from objects in the field, electrical interference from conductive objects in the field, etc.); electrical instrument errors (generally or specifically: shorting, capacitive coupling, broken connections, arcing, other electrical irregularities, etc.); mechanical instrument errors (generally or specifically: insufficient jaw closure, insufficient clamping pressure, damaged tissue-contacting surfaces, etc.); and/or other errors.

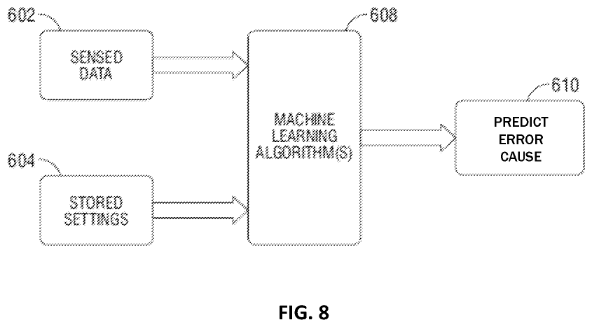

Turning to FIG. 8, in aspects where one or more machine learning machine learning algorithms 608 are used, the memory 46, 426 of the controller 42, 424 of the generator 40, 400 (see FIGS. 4 and 7, respectively) may store the one or more machine learning algorithms 608; alternatively, the one or more one or more machine learning algorithms 608 may be stored on one or more remote computing devices, e.g., servers, accessible by the controller 42, 424 (see FIGS. 4 and 7, respectively). The machine learning algorithm(s) 608 may be trained on and learn from stored settings 604, e.g., theoretical data, empirical data, and/or other data initially input into the one or more machine learning applications, which may include the debug list, and/or sensed data 602 (such as any of the sensed data detailed above) in order to enable the machine learning application(s) to output a predicted cause of the error 610. In aspects, training the machine learning algorithm may be performed by a computing device outside of generator 40, 400 (see FIGS. 4 and 7, respectively) and the resulting algorithm may be communicated to controller 42, 424 of generator 40, 400 (see FIGS. 4 and 7, respectively). In machine learning and/or other aspects, user input may be provided to facilitate identification or errors in future procedures, to train the machine learning algorithms, etc. For example, a user may flag a particular procedure or event therein as successful, unsuccessful; a user may identify an error; a user may identify an optimal result; etc. This may be performed during a digital surgery or other recap of the procedure, for example.

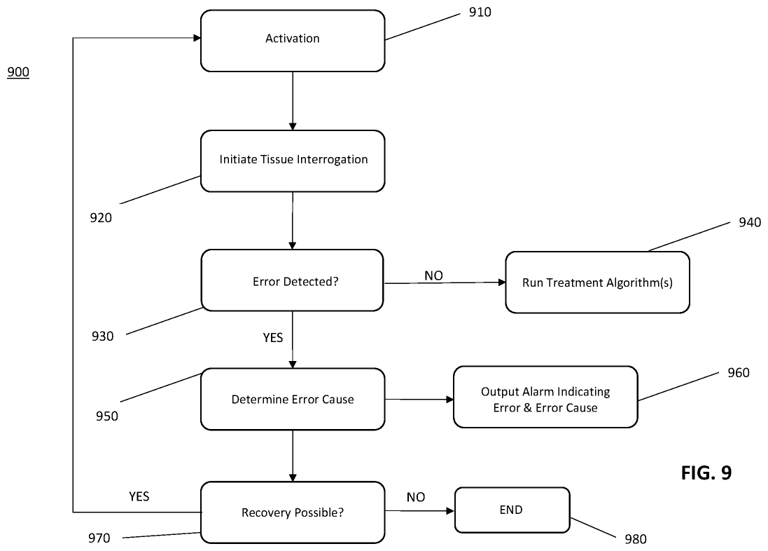

Turning to FIG. 9, a method using an electrosurgical system is provided in accordance with the present disclosure and shown generally identified by reference numeral 900. Method 900 may be performed using any of the electrosurgical instruments, generators, and/or systems detailed herein or with any other suitable electrosurgical instruments, generators, and/or systems. Method 900 begins at 910, upon activation, e.g., when activation switch 30 is activated with tissue grasped between jaw members 110, 120 (FIG. 1).

Once the electrosurgical instrument is activated, tissue interrogation is initiated at 920 and, based on the tissue interrogation (and/or other data), it is determined at 930 whether there is an error. If no error is detected, “NO” at 930, the method proceeds to 940 wherein the tissue treatment algorithm(s), e.g., a pre-treatment and/or tissue sealing algorithm, are run to treat, e.g., seal, tissue. On the other hand, if an error is detected, “YES” at 930, the method proceeds to 950 where the cause of the error is determined, e.g., as detailed above. An alarm (visual, audible, tactile, etc.) is output at 960 (e.g., from the instrument, the generator, and/or another connected device) to alert the user that there is an error, and to inform the user as to the cause of the error. The delivery of energy may also be ceased or otherwise modified when an error is detected.

In addition to outputting an alarm at 960 indicating an error and the cause of the error, or as an alternative thereto, it is determined whether recovery from the error is possible at 970. That is, as noted above, certain detected errors may be cured such as, for example, by adjusting the bite of tissue grasped between the jaw members, by further closing the jaw members, by cleaning/clearing the surgical field, by switching to a different tissue sealing algorithm, by implementing a pre-treatment algorithm before the tissue sealing algorithm, by changing settings of the implemented algorithm, etc. With respect to these or other errors that are recoverable, where it is determined that recovery is possible, “YES” at 970, the method returns to 910 awaiting re-grasp and/or re-activation of the electrosurgical instrument. In aspects, where it is determined that recovery is possible, a recommendation may be output to indicate the step(s) necessary to enable recovery and/or the steps(s) necessary to enable recovery may be automatically implemented, e.g., by update settings, switching the tissue sealing algorithm, implementing a pre-treatment algorithm, etc. With respect to errors that are not recoverable, “NO” at 970, such as, for example, detected instrument electrical/mechanical failure, generator errors, etc., the method ends at 980. Ending the method 980 may disable use of the instrument and/or generator, depending upon the detected error, to ensure safety.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A method of sealing tissue, comprising:
attempting to grasp tissue between first and second jaw members of an end effector assembly of an electrosurgical instrument;
attempting to conduct electrosurgical energy between the first and second jaw members;
determining, based on impedance feedback from the electrosurgical energy, whether an error exists;
in a case where no error is detected, implementing a tissue treating algorithm to treat tissue grasped between the first and second jaw members, wherein the tissue treating algorithm includes conducting electrosurgical energy between the first and second jaw members and through tissue grasped therebetween; and
in a case where an error is detected:
determining, based on additional feedback data, a cause of the error; and
outputting an alarm indicating the error and the cause of the error.

2. The method according to claim 1, wherein the additional feedback data includes electrical feedback data from the electrosurgical energy.

3. The method according to claim 1, wherein the additional feedback data includes sensor data obtained from a sensor.

4. The method according to claim 3, wherein the sensor is configured to one of: sense a physical property of tissue, sense a physical property of the end effector assembly, visualize tissue, or visualize the end effector assembly.

5. The method according to claim 1, wherein determining the cause of the error includes selecting the cause of the error from a debug list based upon the additional feedback data.

6. The method according to claim 5, wherein selecting the cause of the error is performed by a machine learning program.

7. The method according to claim 5, wherein the debug list includes: a tissue grasping error, a field error, and an instrument error.

8. The method according to claim 1, further comprising, in a case where the error is detected, determining, based on the cause of the error, whether the error is recoverable.

9. The method according to claim 8, further comprising:
in a case where the error is determined to be recoverable, enabling electrosurgical energy to be subsequently conducted between the first and second jaw members; and
in a case where the error is determined not to be recoverable, inhibiting the supply of electrosurgical energy to the first and second jaw members.

10. The method according to claim 1, wherein the attempting to conduct the electrosurgical energy between the first and second jaw members includes attempting to conduct an interrogation signal between the first and second jaw members.

11. The method according to claim 10, wherein the interrogation signal includes at least one pulse.

12. The method according to claim 10, wherein the interrogation signal is a constant-power signal.

13. An electrosurgical system, comprising:
an end effector assembly including first and second jaw members, at least one of the first or second jaw members movable relative to the other from a spaced-apart position to an approximated position for grasping tissue therebetween; and
an electrosurgical generator configured to supply electrosurgical energy to the first and second jaw members for conduction therebetween and through grasped tissue to treat the grasped tissue, the electrosurgical generator including a processor and memory storing instructions that, when executed by the processor, cause the processor to:
determine, based on impedance feedback from the electrosurgical energy, whether an error exists;
in a case where no error is detected, implement a tissue treating algorithm to treat tissue grasped between the first and second jaw members, wherein the tissue treating algorithm includes conducting electrosurgical energy between the first and second jaw members and through tissue grasped therebetween; and
in a case where an error is detected:
determine, based on additional feedback data, a cause of the error; and
output an alarm indicating the error and the cause of the error.

14. The electrosurgical system according to claim 13, wherein the end effector assembly is disposed at a distal end of a handheld surgical instrument.

15. The electrosurgical system according to claim 13, wherein the end effector assembly is coupled to a robotic surgical system.

16. The electrosurgical system according to claim 13, wherein the additional feedback data includes electrical feedback data from the electrosurgical energy.

17. The electrosurgical system according to claim 13, wherein the additional feedback data includes sensor data obtained from a sensor associated with the end effector assembly.

18. The electrosurgical system according to claim 13, wherein, in a case where the error is detected, the processor is further caused to:
determine, based on the cause of the error, whether the error is recoverable;
in a case where the error is determined to be recoverable, enabling electrosurgical energy to be subsequently supplied from the electrosurgical generator to the end effector assembly; and
in a case where the error is determined not to be recoverable, inhibiting the supply of electrosurgical energy from the electrosurgical generator to the end effector assembly.

19. The electrosurgical system according to claim 13, wherein the electrosurgical generator is configured to supply electrosurgical energy to the first and second jaw members by way of an interrogation signal to enable the processor to determine whether an error exists.

20. The electrosurgical system according to claim 19, wherein the interrogation signal is at least one of: a pulse signal or a constant-power signal.
